(19) United States
(12) Patent Application Publication
(10) Pub. No.: US 2020/0268703 A1
(43) Pub. Date: Aug. 27, 2020

(54) MEDICINE FOR TREATING ADVANCED OR RECURRENT CANCER PATIENT BEING NON-RESPONSIVE OR NON-TOLERANT TO STANDARD CHEMOTHERAPY AND INCURABLE AND UNRESECTABLE

(71) Applicants: Takahito JOMORI, Nagoya-shi, Aichi (JP); HYOGO COLLEGE OF MEDICINE, Nishinomiya-shi, Hyogo (JP)

(72) Inventors: Shojiro KIKUCHI, Nishinomiya-shi, Hyogo (JP); Mitsuru SASAKO, Nishinomiya-shi, Hyogo (JP); Hiroto MIWA, Nishinomiya-shi, Hyogo (JP); Hisashi SHINOHARA, Nishinomiya-shi, Hyogo (JP); Hiromitsu KISHIMOTO, Nishinomiya-shi, Hyogo (JP); Kazuma NOGUCHI, Nishinomiya-shi, Hyogo (JP); Takahito JOMORI, Nagoya-shi, Aichi (JP)

(21) Appl. No.: 16/495,045

(22) PCT Filed: Mar. 20, 2018
(86) PCT No.: PCT/JP2018/011007

§ 371 Date: Sep. 17, 2019

(30) Foreign Application Priority Data
Mar. 22, 2017 (JP) 2017-055850

(51) Int. Cl. A61K 31/28; A61P 35/04

(52) U.S. Cl. CPC A61K 31/28; A61P 35/04

(57) ABSTRACT

The present invention relates to a medicine for treating a patient with advanced or recurrent cancer which is non-responsive or non-tolerant to standard chemotherapy and incurable and unresectable, the medicine comprising propagermanium.

### TECHNICAL FIELD

The present invention relates to a medicine including propagermanium, which is for treating a cancer patient, particularly, a patient with advanced or recurrent cancer which is non-responsive or non-tolerant to standard chemotherapy and incurable and unresectable.

### BACKGROUND ART

A treatment method for a cancer patient is performed by appropriately combining a surgical treatment, chemotherapy (including immunotherapy), and radiation therapy, according to a type of cancer, a degree of progression (stage), and a morbid state and a condition of a patient. In recent years, a treatment guideline has been established for each type of cancer. The chemotherapy is positioned as preoperative and postoperative adjuvant chemotherapy, induction chemotherapy performed prior to a treatment, standard therapy for unresectable advanced or recurrent cancer, or the like.

However, for advanced or recurrent cancer which cannot be resected and for which there is almost no therapeutic effect from chemotherapy, there is an only way to do palliative care (best supportive care; BSC) to reduce pain, and currently there is no fundamental treatment method.

On the other hand, propagermanium is a kind of an organic germanium compound, and has been marketed in Japan as a medicine for chronic hepatitis B. Regarding anticancer activity of organic germanium compound, Patent Literature 1 describes that the organic germanium compound is effective against various cancers such as gastric cancer and lung cancer. However, there is no specific disclosure based on pharmacological data on efficacy of propagermanium against gastric cancer or head and neck cancer. Furthermore, there is no disclosure of any specific method of identifying a cancer patient who can expect therapeutic effects by administration of the propagermanium.

### CITATION LIST

### Patent Literature

[Patent Literature 1] Japanese Unexamined Patent Publication No. S59-16825

### SUMMARY OF INVENTION

### Technical Problem

An object of the present invention is to provide a medicine for treating a identified cancer patient, which includes propagermanium as an active ingredient, and a method of evaluating a therapeutic effect in the cancer patient.

### Solution to Problem

As a result of conducting intensive study to achieve the object, the present inventors have found that propagermanium is useful for treatment of a patient with advanced or recurrent cancer which is non-responsive or non-tolerant to standard chemotherapy and incurable and unresectable, and is useful for treatment of a cancer patient showing a specific index value with experimental administration of the propagermanium and have completed the present invention.

That is, the present invention provides the following aspects [1] to [16].

[1] A medicine for treating a patient with advanced or recurrent cancer which is non-responsive or non-tolerant to standard chemotherapy and incurable and unresectable, the medicine comprising: propagermanium.
[2] The medicine according to [1], wherein the cancer patient is an adenocarcinoma patient.
[3] The medicine according to [2], wherein the adenocarcinoma patient is a patient with one or more kinds of cancer selected from the group consisting of gastric cancer, biliary tract cancer, liver cancer, pancreatic cancer, prostate cancer, breast cancer, ovarian cancer, colon cancer, and rectal cancer.
[4] The medicine according to [2], wherein the adenocarcinoma patient is a gastric cancer patient.
[5] The medicine according to [4], wherein the standard chemotherapy is standard chemotherapy using one or more chemotherapeutic agents selected from the group consisting of S-1, capecitabine, 5-fluorouracil, cisplatin, oxaliplatin, docetaxel, paclitaxel, irinotecan, trastuzumab, and ramucirumab.
[6] The medicine according to [1], wherein the cancer patient is a squamous cell carcinoma patient.
[7] The medicine according to [6], wherein the squamous cell carcinoma patient is a patient with one or more kinds of cancer selected from the group consisting of oral cavity cancer, pharyngeal cancer, laryngeal cancer, thyroid cancer, salivary gland cancer, esophageal cancer, lung cancer, skin cancer, and uterine cancer.
[8] The medicine according to [6], wherein the squamous cell carcinoma patient is an oral cavity cancer patient.
[9] The medicine according to [8], wherein the standard chemotherapy is standard chemotherapy using one or more chemotherapeutic agents selected from the group consisting of S-1, 5-fluorouracil, cisplatin, carboplatin, nedaplatin, docetaxel, paclitaxel, and cetuximab.
[10] The medicine according to any one of [1] to [9], wherein the cancer patient is a patient whose ECOG performance status is 0 or 1.
[11] The medicine according to any one of [1] to [10], wherein the cancer patient is a patient without peritoneal metastasis.
[12] The medicine according to any one of [1] to [11], wherein the cancer patient is a patient whose a tumor immunity risk index value after completion of experimental administration of the propagermanium is 3.5 or less or shows a numerical value less than the tumor immunity risk index value before the experimental administration of the propagermanium.
[13] A medicine for treating a cancer patient whose a tumor immunity risk index value after completion of experimental administration of propagermanium is 3.5 or less or shows a numerical value less than the tumor immunity risk index value before the experimental administration of the propagermanium, the medicine comprising: propagermanium.
[14] A method for treating a patient with advanced or recurrent cancer which is non-responsive or non-tolerant to standard chemotherapy and incurable and unresectable, the method comprising: administering propagermanium to the patient in need thereof.
[15] Use of propagermanium for treating a patient with advanced or recurrent cancer which is non-responsive or non-tolerant to standard chemotherapy and incurable and unresectable.
[16] Use of propagermanium for producing a medicine for treating a patient with advanced or recurrent cancer which is non-responsive or non-tolerant to standard chemotherapy and incurable and unresectable.

### Advantageous Effects of Invention

According to the present invention, it is possible to provide a medicine for treating a patient with advanced or recurrent cancer which is non-responsive or non-tolerant to standard chemotherapy and incurable and unresectable. Also, according to the present invention, it is possible to provide a medicine which includes propagermanium and is for treating a cancer patient whose therapeutic effect therefrom is expected. Furthermore, according to the present invention, it is possible to provide a method of evaluating a therapeutic effect, by a medicine including propagermanium in a cancer patient.

### BRIEF DESCRIPTION OF DRAWINGS

FIG. 1 is a graph showing overall survival (OS) of 62 patient cases in which palliative care is performed and overall survival (OS) of 13 patient cases in which propagermanium is administered, as Kaplan Meier survival curves in an example.

FIG. 2 is a graph showing overall survival (OS) of patient cases in which palliative care is performed and overall survival (OS) of patient cases in which propagermanium is administered, in a patient group whose ECOG performance status (PS) is 0 or 1, as Kaplan Meier survival curves in an example.

FIG. 3 is a graph showing overall survival (OS) of patient cases in which palliative care is performed and overall survival (OS) of patient cases in which propagermanium is administered, in a patient group whose ECOG performance status (PS) is 2 or 3, as Kaplan Meier survival curves in an example.

FIG. 4 is a graph comparing mean values of overall survival (OS) according to presence or absence of peritoneal metastasis, in each group of all patients, a patient group whose ECOG performance status (PS) is 0 and 1, and a patient group whose ECOG performance status (PS) is 2 and 3, among 13 patient cases in which propagermanium is administered, in an example.

FIG. 5 shows photographs obtained by imaging a lesion site of lung of a patient P-5, whose lung metastasis has been observed before administration of propagermanium, at the time of starting administration of the propagermanium and on 35th, 85th, and 357th days after starting the administration, in an example. An arrow in the photograph indicates the lesion site.

FIG. 6 is a graph showing apoptosis-inducing activity by peripheral blood mononuclear cells (PBMC) of an oral cavity cancer patient after the administration of propagermanium, in an example.

### DESCRIPTION OF EMBODIMENTS

Hereinafter, the present invention will be described.

[1. Invention of Medicine for Treating Cancer Patient, Including Propagermanium]

A medicine according to the present invention comprises propagermanium as an active ingredient. The propagermanium is an organic germanium compound represented by the following formula.

[(O_{1/2})₃GeCH₂CH₂COOH]ₙ

(In the formula, n represents an integer of 200 to 900)

The propagermanium is represented by the following structural formula.

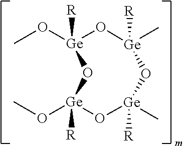

[In the formula, R represents —CH₂CH₂COOH, m represents a weight average polymerization degree converted from a weight average molecular weight of a 3-oxygermylpropionic acid propyl ester polymer, and is an integer of 137±84 (mean value±standard error 3σ))]

The minimum constitutional unit is (O_{1/2})₃GeCH₂CH₂COOH and an experimental formula is preferably a 8-membered ring structure (J. Pharm. Sci., 104, 2482-2488, 2015) represented by C₆H₁₀Ge₂O₇. The propagermanium can be produced by a method described in Japanese Unexamined Patent Publication No. 2003-81843 and the like.

According to the present invention, there is provided a medicine for treating a patient with advanced or recurrent cancer which is non-responsive or non-tolerant to standard chemotherapy and incurable and unresectable, the medicine comprising propagermanium as the active ingredient.

In the present specification, the term “standard chemotherapy” refers to chemotherapy for various cancer patients standardized according to race and the like, in each country or region. At present, there is a possibility that latest standard chemotherapy may be revised and/or changed by development of a new medicine or the like. However, the standard chemotherapy is set to include both of the standard chemotherapy before and after the revision and/or the change. The standard chemotherapy includes immunotherapy.

For example, as the latest standard chemotherapy of gastric cancer in Japan, there is chemotherapy described in FIG. 8 (an algorithm of chemotherapy for advanced and recurrent gastric cancer which is unresectable) in gastric cancer treatment guidelines, fourth edition (edited by the Japan gastric cancer society, edited in May 2014) of Japanese Gastric Cancer Association. The standard chemotherapy in Japan includes chemotherapy for HER2-negative gastric cancer and chemotherapy for HER2-positive gastric cancer. Here, the HER2 refers to human epidermal growth factor receptor 2.

Specifically, the chemotherapy for HER2-negative gastric cancer in Japan is a treatment method of performing each of combined administration of S-1 (or capecitabine) and cisplatin (or oxaliplatin) in a first-line treatment, administration of docetaxel or paclitaxel alone in a second-line treatment, and administration of irinotecan alone in a third-line treatment or a treatment method of performing each of combined administration of S-1 (or capecitabine) and cisplatin (or oxaliplatin) in a first-line treatment, administration of irinotecan alone in a second-line treatment, and administration of docetaxel or paclitaxel alone in a third-line treatment. Here, “S-1” (also referred to as “TS-1”) is a medicine comprising tegafur, gimeracil, and oteracil potassium, as active ingredients.

Specifically, the chemotherapy for HER2-positive gastric cancer in Japan is a treatment method of performing each of combined administration of capecitabine (or S-1), cisplatin (or oxaliplatin), and trastuzumab in a first-line treatment, administration of docetaxel or paclitaxel alone in a second-line treatment, and administration of irinotecan alone in a third-line treatment or a treatment method of performing each of combined administration of capecitabine (or S-1), cisplatin (or oxaliplatin), and trastuzumab in a first-line treatment, administration of irinotecan alone in a second-line treatment, and administration of docetaxel or paclitaxel alone in a third-line treatment.

As the latest standard chemotherapy for gastric cancer in United States, there is chemotherapy described in “Systematic treatment method of unresectable local, recurrent, metastatic gastric cancer” GAST-F (3 of 11) in gastric cancer treatment guidelines (J. Natl. Compr. Cane. Netw., Vol. 14(19), 1286-1312, 2016) of National Comprehensive Cancer Network. Specifically, a treatment method of performing each of combined administration of fluoropyrimidine (5-fluorouracil or capecitabine) and cisplatin (or oxaliplatin) in a first-line treatment and combined administration of ramucirumab and paclitaxel or administration of docetaxel, paclitaxel, irinotecan, or ramucirumab alone in a second-line treatment is recommended.

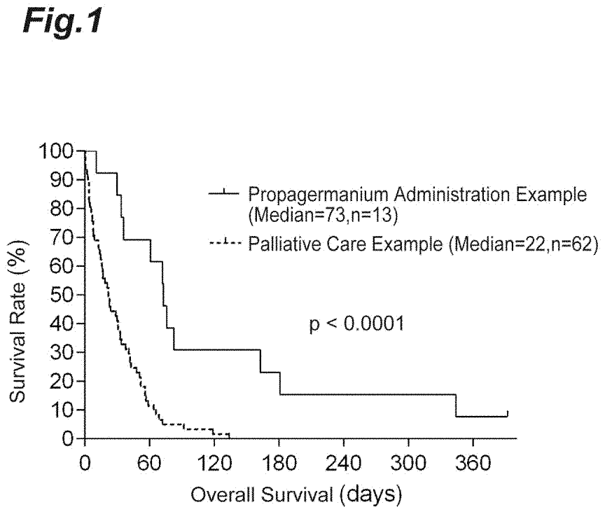

As the latest standard chemotherapy of gastric cancer in Europe, there is chemotherapy described in “Unresectable or metastatic” in FIG. 1 (Treatment algorithm for gastric cancer) in gastric cancer treatment guidelines (Ann. Oncol., Vol. 27 (Suppl. 5), v 38-v49, 2016) of European Society for Medical Oncology. The standard chemotherapy in Europe includes chemotherapy for HER2-negative gastric cancer, chemotherapy for HER2-positive gastric cancer, and an examination of clinical trial of a new medicine.

Specifically, the chemotherapy for HER2-negative gastric cancer in Europe is a treatment method of using a platinum preparation and fluoropyrimidine in combination. Specifically, the chemotherapy for HER2-positive gastric cancer in Europe is a treatment method using trastuzumab, cisplatin, and 5-fluorouracil or capecitabine in combination.

When summarizing the latest standard chemotherapy for gastric cancer in each country or region exemplified above, the standard chemotherapy can be described as “standard chemotherapy using one or more chemotherapeutic agents selected from the group consisting of S-1, capecitabine, 5-fluorouracil, cisplatin, oxaliplatin, docetaxel, paclitaxel, irinotecan, trastuzumab, and ramucirumab”.

For example, the latest standard chemotherapy of head and neck cancer in Japan refers to chemotherapy for 2. Recurrence/Metastasis in III-A-2. Cancer pharmacotherapy of head and neck cancer treatment guidelines 2018 edition (edited by the Japan Head and Neck Cancer Society, Kinbara publication) of Japan Head and Neck Cancer Society. Specific examples of a treatment method for recurrent cancer patient after initial treatment include a treatment method of performing administration of a platinum preparation alone, combined administration of a platinum preparation and 5-fluorouracil, and a combination of a platinum preparation, 5-fluorouracil, and cetuximab. Here, examples of the platinum preparation can include cisplatin and carboplatin. Specific examples of a treatment method for cases non-responsive to the platinum preparation include a treatment method of performing administration of docetaxel, paclitaxel, S-1, or nivolumab alone. In recent years, nedaplatin, which was created for the purpose of reducing nephrotoxicity of cisplatin, is also used.

As the latest standard chemotherapy for head and neck cancer in United States, there is chemotherapy described in “Principle of systematic treatment method, recurrent/unresectable/metastatic” in CHEM-A 2 OF 5 in head and neck cancer treatment guidelines (J. Natl. Compr. Canc. Netw., Vol. 15(6), 761-770, 2017) of National Comprehensive Cancer Network.

As the standard chemotherapy for head and neck cancer in United States, there is combination therapy and monotherapy. Specific examples of the combination therapy include combined administration of cisplatin or carboplatin and 5-fluorouracil and cetuximab, combined administration of cisplatin or carboplatin and docetaxel or paclitaxel, combined administration of cisplatin and cetuximab, combined administration of cisplatin and 5-fluorouracil, combined administration of cisplatin or carboplatin and docetaxel and cetuximab, combined administration of cisplatin or carboplatin and paclitaxel and cetuximab, combined administration of carboplatin and cetuximab, combined administration of cisplatin and gemcitabine, and combined administration of gemcitabine and vinorelbine. Specific examples of the monotherapy include administration of cisplatin, carboplatin, paclitaxel, docetaxel, 5-fluorouracil, methotrexate, cetuximab, gemcitabine, capecitabine, afatinib, pembrolizumab, or nivolumab alone.

As the latest standard chemotherapy of head and neck cancer in Europe, there is chemotherapy described in “treatment of recurrent or metastatic disease” in head and neck cancer (squamous cell carcinoma) treatment guidelines (Ann. Oncol., Vol. 21 (Supp1.5), v184-v186, 2010) and “local, regional and metastasis recurrence” in head and neck cancer (epipharyngeal cancer) treatment guidelines (Ann. Oncol., Vol. 23 (Suppl. 7), vii 83-vii85, 2012) of European Society for Medical Oncology.

Specific examples of standard chemotherapy for the head and neck cancer (epipharyngeal cancer) in Europe include a treatment method of administration of cisplatin, paclitaxel, docetaxel, gemcitabine, capecitabine, irinotecan, vinorelbine, ifosfamide, doxorubicin, or oxaliplatin alone, and a treatment method of combined administration of cisplatin and a drug other than the cisplatin.

In the standard chemotherapy for the head and neck cancer (squamous cell carcinoma) in Europe, specifically, combined administration of cisplatin or carboplatin and cetuximab and 5-fluorouracil is recommended for combination therapy, and administration of methotrexate or cetuximab alone is recommended for monotherapy.

When summarizing the latest standard chemotherapy for head and neck cancer in each country or region exemplified above, the standard chemotherapy can be described as “standard chemotherapy using one or more chemotherapeutic agents selected from the group consisting of S-1, 5-fluorouracil, cisplatin, carboplatin, nedaplatin, docetaxel, paclitaxel, and cetuximab”.

In the present specification, the term “non-responsive or non-tolerant to standard chemotherapy” refers to a case where a therapeutic effect was not recognized although the standard chemotherapy was performed, a case where a drug cannot be administered due to deterioration of a condition or an adverse event, a case where the standard chemotherapy cannot be performed due to a morbid state of a patient from the beginning, a case where a treatment by the standard chemotherapy is extremely difficult or practically impossible.

In Experimental Example 6 to be described below, since it has been confirmed that the medicine of the present invention enhances apoptosis induction ability of peripheral blood lymphocytes of an oral cavity cancer patient who received the medicine of the present invention by administration, to gastric cancer cells, compared to before the administration, it is considered to exhibit anti-tumor activity nonspecifically to various cancer cells. Therefore, various cancer patients can be targeted for treatment. In the medicine of the present invention, examples of the cancer patient who can be targeted for treatment include a gastric cancer patient, a biliary tract cancer patient, a colorectal cancer patient, a liver cancer patient, a pancreatic cancer patient, a renal cancer patient, a prostate cancer patient, a testicular cancer patient, a bladder cancer patient, a breast cancer patient, an ovarian cancer patient, a colon cancer, a rectal cancer, a head and neck cancer patient (an oral cavity cancer (tongue cancer) patient, a maxillary sinus cancer patient, a pharyngeal cancer (epipharyngeal cancer, oropharyngeal cancer, and hypopharyngeal cancer) patient, a laryngeal cancer patient, a thyroid cancer patient, a salivary gland cancer (parotid gland cancer and submaxillary gland cancer) patient), an esophageal cancer patient, a lung cancer patient, a skin cancer patient, a uterine cancer patient, a brain tumor patient, a multiple myeloma patient, a sarcoma patient, a malignant lymphoma patient, and a leukemia patient. Among these, solid cancer patients are preferable, and an adenocarcinoma patient or a squamous cell carcinoma patient is more preferable.

According to an aspect of the present invention, the cancer patient who is targeted for treatment is the adenocarcinoma patient. Among which, a cancer patient with gastric cancer, biliary tract cancer, liver cancer, pancreatic cancer, prostate cancer, breast cancer, ovarian cancel; colon cancer, or rectal cancer is preferable, and a gastric cancer patient is more preferable. Also, according to another aspect of the present invention, the cancer patient who is targeted for treatment is a squamous cell carcinoma patient. Among which, a cancer patient with oral cavity cancer (tongue cancer), pharyngeal cancer (epipharyngeal cancer, oropharyngeal cancer, and hypopharyngeal cancer), laryngeal cancer, thyroid cancer, salivary gland cancer (parotid gland cancer and submaxillary gland cancer), esophageal cancer, lung cancer, skin cancer, or uterine cancer is preferable, and an oral cavity cancer patient is more preferable.

In the present specification, the term “ECOG performance status” means that an activity of cancer patients is defined according to scores by Eastern Cooperative Oncology Group (ECOG). Specifically, the scores of the ECOG performance status are as follows.

0: It is possible to act without any problem. The same daily life as before the onset of disease can be performed without restriction.

1: Physically intense action is restricted, but it is possible to walk and do light work and sitting work (for example, light housework or office work).

2: It is possible to walk and do everything around themselves, but working is not possible. Over 50% of the daytime is spent outside a bed.

3: It is possible to only do restricted things around themselves. More than 50% of the daytime is spent in a bed or a chair.

4: It is not possible to move at all. It is not possible to do everything around themselves. Spending completely in a bed or in a chair.

The medicine according to the present invention is more effective for a patient whose ECOG performance status is 0 or 1, who is possible to at least lightly act, among patients with advanced or recurrent cancer which is non-responsive or non-tolerant to standard chemotherapy and incurable and unresectable, and is preferably used for the treatment of these patients. The patients whose the ECOG performance status is 0 or 1 can be identified by a diagnosis or the like of a doctor.

In the present specification, the term “peritoneal metastasis” refers to metastasis of cancer cells to peritoneum. The medicine according to the present invention is more effective for a patient without peritoneal metastasis, among the patients with advanced or recurrent cancer which is non-responsive or non-tolerant to standard chemotherapy and incurable and unresectable, and is preferably used for the treatment of these patients. In addition, the medicine according to the present invention is particularly effective for a patient whose the ECOG performance status is 0 or 1, and who is without the peritoneal metastasis, among the patients with advanced or recurrent cancer which is non-responsive or non-tolerant to standard chemotherapy and incurable and unresectable, and it is particularly preferable to use the medicine for the treatment of these patients. The patients without the peritoneal metastasis can be identified by a diagnosis of a doctor or the like.

Among the cancer patients, the medicine according to the present invention is preferably used for treating a patient whose a tumor immunity risk index value after completion of experimental administration of propagermanium is 3.5 or less (preferably 3.0 or less) or shows a numerical value less than the tumor immunity risk index value before the experimental administration of the propagermanium, among the cancer patients. The patient can be identified by performing a calculation step and an evaluation step in an evaluation method described in [2. Invention of Method of Evaluating Therapeutic Effect Obtained by Medicine Comprising Propagermanium in Cancer Patient] to be described later.

Here, the term “experimental administration” means that a few times of administration are performed to predict efficacy of the medicine. Administration period in the experimental administration can be appropriately set according to a state of a patient and circumstances, but is set preferably as a period of approximately 20 days to 50 days, more preferably as a period of approximately 25 days to 45 days, and still more preferably as a period of approximately 28 days to 42 days.

That is, according to still another aspect of the present invention, there is provided a medicine for treating a cancer patient whose a tumor immunity risk index value after completion of experimental administration of propagermanium is 3.5 or less (preferably 3.0 or less) or shows a numerical value less than the tumor immunity risk index value before the experimental administration of the propagermanium, the medicine comprising propagermanium as an active ingredient.

When the medicine according to the present invention is used as a solid preparation for oral use, the medicine can be formed into a tablet, a coated tablet, granules, fine granules, a powdered medicine, a capsule, and the like by a usual method. Also, in addition to sugar coating and gelatin coating, the medicine can be coated as needed, as long as a substance and a shape that can be used for medical supplies are used. Furthermore, pharmaceutically acceptable additives such as an excipient, a binder, a disintegrant, a lubricant, a coloring agent, and a flavoring agent can be appropriately combined, as needed. As the excipient, saccharides such as lactose, sucrose, and dextrans; a cellulose-based polymeric substance such as hydroxypropyl cellulose; and naturally-occurring polymeric substance such as albumin can be used.

In addition to the medicine according to the present invention can be administered, for example, orally as a solution or syrup, or parenterally as an injection, eye drop, nasal drop, a suppository, or ointment, in addition to the solid preparation for oral use by common preparation techniques. Formulation of the medicine according to the present invention can be performed according to a method described in Japanese Unexamined Patent Publication No. 2003-81843 and the like.

A dose and the number of doses when the medicine according to the present invention is administered to a human can be appropriately set according to a dosage form, or a condition, age, sex, and the like of a patient. For example, for oral administration to an adult (body weight 60 kg), 1 to 1000 mg, preferably 5 to 500 mg, and more preferably 10 to 120 mg can be administered once or in several divided doses per day.

[2. Invention of Method of Evaluating Therapeutic Effect Obtained by Medicine Comprising Propagermanium in Cancer Patient]

An evaluation method of the present invention is a method of evaluating a therapeutic effect obtained by a medicine comprising propagermanium in a cancer patient, and comprises the calculation step of calculating a tumor immunity risk index value in the cancer patient at each time of before administration of the propagermanium and after completion of the administration and the evaluation step of evaluating the therapeutic effect of the medicine in the cancer patient based on a result obtained by the calculation in the calculation step.

In the calculation step, the tumor immunity risk index value in the cancer patient at each time of before administration of the propagermanium and after completion of the administration is calculated. A calculation method therefor is as follows.

A circulating tumor cell (circulation tumor cell; CTC) in blood is a tumor cell released from a primary tumor tissue or a metastatic tumor tissue and infiltrated into the blood. A measurement of the number of the circulating tumor cells in the blood is regarded as a direct evaluation method of a cancer diagnosis or a cancer therapeutic effect, and measurement methods using various techniques have been developed. In most of existing methods of measuring the circulating tumor cell in the blood, EpCAM (CD326) in cancer cells is used as a marker. It is known that expression of the EpCAM is attenuated or eliminated in many high-grade circulating tumor cells in blood. Therefore, existing measurement method has a problem that the high-grade circulating tumor cells in blood cannot be detected. On the other hand, in a method of combining a plurality of intracellular keratin isoforms, although most circulating tumor cells in blood can be detected, there is a problem that a false positive rate increases. The present inventors have found that most circulating tumor cells in blood can be detected with high accuracy when using pankeratin antibodies that recognize keratins 4, 5, 6, 8, 10, 13, and 18 to measure pankeratinocytes in which these keratins are expressed in cells. On the other hand, it was clear that cytotoxic T cells play an important role in tumor immunity as a factor on a host side. Therefore, the present inventors considered that the circulating tumor cells in blood and a proportion of the cytotoxic T cells in T cells of the cancer patient is a risk index in tumor immunity, and defined a numerical value calculated by Expression (I) below as the “tumor immunity risk index value”.

Number of pankeratin positive cells (pieces)/Proportion of cytotoxic T cells in total T cells (%)/10 (I)

It can be evaluated that the greater the tumor immunity risk index value, the higher the risk in the tumor immunity. Although there are individual differences, the tumor immunity risk index value in a healthy person is usually less than 1.0.

In Expression (I), the term “Pankeratin positive cells” refers to cells positive for keratin 4, 5, 6, 8, 10, 13, and 18 and negative for CD45.

In Expression (I), the term “Number of pankeratin positive cells” is the number of pankeratin positive cells in 10000 peripheral blood mononuclear cells (PBMCs). The number of the pankeratin positive cells can be calculated in a manner that the peripheral blood mononuclear cells are prepared by a usual method from peripheral blood collected from a cancer patient, an inside of the obtained peripheral blood mononuclear cells is labeled with a pankeratin antibody, and the labeled peripheral blood mononuclear cells are analyzed by flow cytometry to select pankeratin positive cells.

In Expression (I), the term “T cell” refers to a lymphocyte having a T cell receptor, and is synonymous with “CD45 positive and CD3 positive cell”. In Expression (I), the term “Cytotoxic T cells” refers to T cells having cell killing activity, and is synonymous with “CD8 positive and CD4 negative cells”.

In Expression (I), the term “proportion of cytotoxic T cells in total T cells” refers to a value expressing a proportion of the cytotoxic T cells when the total number of T cells in blood is 100%, by a percentage. The total number of the T cells or the number of cytotoxic T cells can be calculated in a manner that, for example, the peripheral blood mononuclear cells are prepared by a usual method from peripheral blood collected from a cancer patient, a surface of the obtained peripheral blood mononuclear cells is labeled with a T cell staining antibody, the fluorescently labeled peripheral blood mononuclear cells are analyzed by flow cytometry, and a population of CD45 positive and CD3 positive cells or a population of CD8 positive and CD4 negative cells is selected.

In the evaluation step, based on the result obtained by the calculation in the calculation step, the therapeutic effect of the medicine comprising propagermanium in the cancer patient is evaluated. Specifically, when the tumor immunity risk index value of the cancer patient after the propagermanium is administered for a certain period of time is 3.5 or less (preferably 3.0 or less) or shows a numerical value less than the tumor immunity risk index value before the administration of the propagermanium, it is evaluated that a therapeutic effect can be expected. Here, when the time after the propagermanium is administered for a certain period of time is the time after the experimental administration, it is possible to predict efficacy of previous administration of the propagermanium. When the time after the propagermanium is administrated for a certain period of time is the time after the completion of usual administration, it is possible to determine whether the administration until then was effective or not, without performing other inspections.

In the evaluation method according to the present invention, a kind of cancer for which a therapeutic effect can be evaluated is not limited, and it is possible to evaluate the therapeutic effect in a cancer patient with gastric cancer, biliary tract cancer, colorectal cancer, liver cancer, pancreatic cancer, renal cancer, prostate cancer, testicular cancer, bladder cancer, breast cancer, ovarian cancer, colon cancer, rectal cancer, head and neck cancer (oral cavity cancer (tongue cancer), maxillary sinus cancer, pharyngeal cancer (epipharyngeal cancer, oropharyngeal cancer, and hypopharyngeal cancer), laryngeal cancer, thyroid cancer, salivary gland cancer (parotid gland cancer and submaxillary gland cancer), esophageal cancer, lung cancer, skin cancer, uterine cancer, brain tumor, multiple myeloma, sarcoma, malignant lymphoma, or leukemia. Among these, the evaluation method according to the present invention is preferably used to evaluate the therapeutic effect on a solid cancer patient, and more preferably, to evaluate the therapeutic effect on an adenocarcinoma patient or a squamous cell carcinoma patient. According to an aspect of the present invention, the evaluation method is preferably used to evaluate the therapeutic effect on the adenocarcinoma patient. Among the adenocarcinoma patients, the evaluation method is preferably used to evaluate the therapeutic effect on a patient with one or more cancer selected from the group consisting of gastric cancer, biliary tract cancer, liver cancer, pancreatic cancer, prostate cancer, breast cancer, ovarian cancer, colon cancer, and rectal cancer, more preferably used to evaluate the therapeutic effect on a gastric cancer patient, and still more preferably used to evaluate the therapeutic effect on a patient with advanced or recurrent gastric cancer which is non-responsive or non-tolerant to standard chemotherapy and incurable and unresectable. In addition, according to another aspect of the present invention, the evaluation method is preferably used to evaluate the therapeutic effect on the squamous cell carcinoma patient. Among the squamous cell carcinoma patient, the evaluation method is preferably used to evaluate therapeutic effect on a patient with one or more cancer selected from the group consisting of oral cavity cancer (tongue cancer), pharyngeal cancer (epipharyngeal cancer, oropharyngeal cancer, and hypopharyngeal cancer), laryngeal cancer, thyroid cancer, salivary gland cancer (parotid gland cancer and submaxillary gland cancer), esophageal cancer, lung cancer, skin cancer, and uterine cancer, and more preferably used to evaluate the therapeutic effect on the oral cavity cancer patient.

### EXAMPLES

Hereinafter, the present invention will be described in detail with reference to Examples, but the present invention is not limited to the following Examples.

### <Experimental Example 1> Therapeutic Effect of Propagermanium Administration on Advanced or Recurrent Gastric Cancer which is Non-responsive or Non-tolerant to Standard Chemotherapy and Incurable and Unresectable

30 mg of propagermanium [trade name: SEROCION (registered trademark), produced by SANWA KAGAKU KENKYUSHO CO., LTD.] was administered orally in three divided doses per day after meals to 13 patient cases (P-1 to P-13) who were diagnosed as advanced or recurrent gastric cancer which is non-responsive or non-tolerant to standard chemotherapy [gastric cancer treatment guideline fourth edition (edited by Japan Gastric cancer Association, revised in May 2014)] and incurable and unresectable, and a clinical trial was conducted with overall survival (hereinafter also referred to as “OS”) and progression-free survival (hereinafter also referred to as “PFS”) as evaluation items. This clinical trial was conducted under an approval of Ethics Committee of Hyogo College of Medicine (No. 1911). The ECOG performance status (hereinafter also referred to as “PS”), the OS, and the PFS of 13 patient cases are shown in Table 1.

TABLE 1

| Patient | PS | OS (day) | PFS (day) |
|---|---|---|---|
| P-1 | 2 | 76 | 56 |
| P-2 | 3 | 34 | 3 |
| P-3 | 0 | 344 | 336 |
| P-4 | 2 | 72 | 28 |
| P-5 | 1 | 392 | 392 |
| P-6 | 3 | 36 | 22 |
| P-7 | 0 | 83 | 49 |
| P-8 | 1 | 73 | 73 |
| P-9 | 3 | 30 | 21 |
| P-10 | 2 | 11 | 7 |
| P-11 | 0 | 163 | 159 |
| P-12 | 1 | 181 | 178 |
| P-13 | 1 | 61 | 36 |

| | | |
|---|---|---|
| Median | 73 | 49 |

A median of the OS of the 13 patient cases in which the propagermanium was administered was 73 days, and a median of PFS thereof was 49 days. Patient P-5 is alive at 392nd days after starting the administration of the propagermanium.

On the other hand, in Hyogo College of Medicine Hospital from 2009 to 2011, in 62 cases which were diagnosed as advanced gastric cancer which is non-responsive or non-tolerant to standard chemotherapy and incurable and unresectable and for which palliative care (Best Supportive Care; hereinafter “BSC”) to reduce pain was provided, the median of OS from the time when a doctor explained BSC to the patient was 22 days. In addition, in Kaplan Meier survival curves of the OS of the BSC example and the propagermanium administration example shown in FIG. 1, a significant life prolongation effect by the administration of the propagermanium was observed.

### <Experimental Example 2> Comparison Test of Overall Survival Using Difference of PS

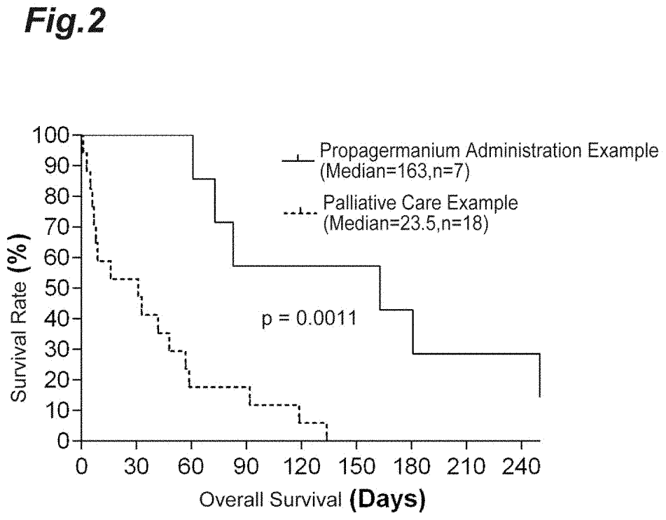

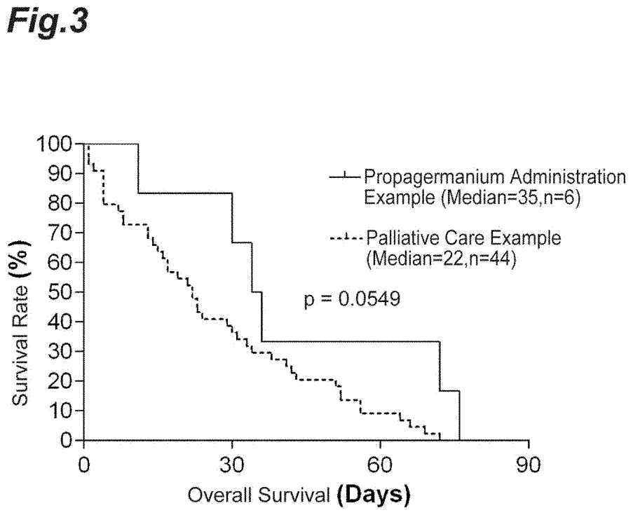

13 Patient cases in which the propagermanium was administered and 62 patient cases in which the BSC was provided were divided into two groups of (A) 0 and 1 and (B) 2 and 3 according to PS thereof. In the two groups, medians of the OS in the propagermanium administration examples and BSC examples were compared. As a result, in the patient groups whose PS was (A) 0 and 1, the median of OS in the BSC examples was 23.5 days, whereas the median of OS in the propagermanium administration examples was 163 days. The survival time was extended approximately 7 times compared to the BSC examples. In addition, also in the KaplanMeier survival curve shown in FIG. 2, a significant life prolongation effect by the administration of the propagermanium was observed in the patient groups whose PS was (A) 0 and 1. On the other hand, as shown in FIG. 3, in the patient groups whose PS is (B) 2 and 3, the median of OS of the BSC examples was 22 days, whereas the median of OS of the propagermanium administration examples was 35 days. Accordingly, it was confirmed that the survival days were extended compared to the BSC examples. In addition, also in Kaplan Meier survival curve of the drawing, although significant difference was not confirmed, a tendency to prolong life by the administration of the propagermanium was observed.

### <Experimental Example 3> Comparison Test of Overall Survival Using Presence or Absence of Peritoneal Metastasis

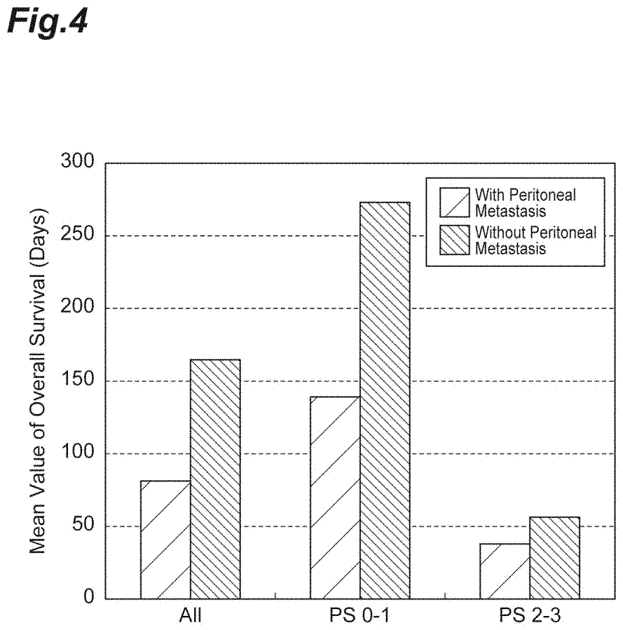

In 13 patient cases in which the propagermanium was administered, mean values of OS were compared according to the presence and absence of the peritoneal metastasis (Table 2 and FIG. 4). As a result, the mean value of OS of 8 patient cases with the peritoneal metastasis was 81.1 days, whereas the mean value of OS of 5 patient cases without the peritoneal metastasis was 164.7 days. A significant life prolongation effect was observed in the patient cases without the peritoneal metastasis. In addition, 13 patient cases in which the propagermanium was administered were divided into two groups of (C) 0 and 1 and (D) 2 and 3 according to the PS thereof. In the two groups, mean values of the OS were compared according to the presence or absence of the peritoneal metastasis (Table 2 and FIG. 4). As a result, in the patient group whose PS was (C) 0 and 1, the mean value of OS of 4 cases with the peritoneal metastasis was 139 days, whereas the mean value of OS of 3 cases without the peritoneal metastasis was 273 days. Therefore, a significant life prolongation effect was observed in patient cases without the peritoneal metastasis. On the other hand, in the patient group whose PS was (D) 2 and 3, the mean value of OS of 4 cases with the peritoneal metastasis was 37.8 days, whereas the mean value of OS of 2 cases without the peritoneal metastasis was 56.3 days. Although significant difference was not confirmed, a tendency to prolong life was observed in the patient cases without the peritoneal metastasis.

TABLE 2

| | With peritoneal | Without peritoneal |
|---|---|---|
| | metastasis (number | metastasis (number |
| | of cases) | of cases) |
| All patient | 81.1 (8) | 164.7 (5) |
| (C) Patient whose | 139 (4) | 273 (3) |
| PS is 0 and 1 | | |
| (D) Patient whose | 37.8 (4) | 56.3 (2) |
| PS is 2 and 3 | | |

### <Experimental Example 4> Effect of Propagermanium Administration on Patient with Lung Metastasis Foci in Advanced or Recurrent Gastric cancer which is Non-responsive or Non-tolerant to Standard Chemotherapy and Incurable and Unresectable

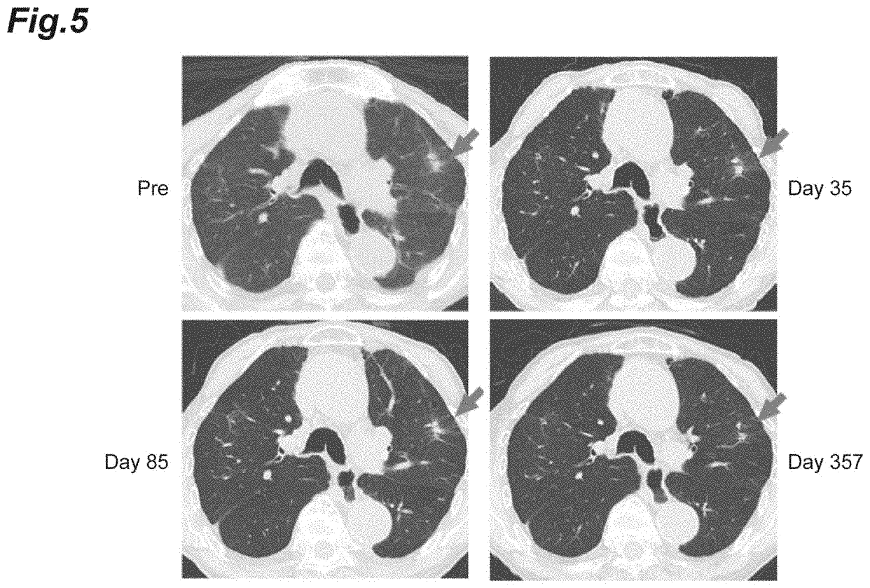

FIG. 5 shows photographs obtained by imaging a lesion site of lung of the patient P-5, whose lung metastasis has been observed before administration of the propagermanium, in 13 patient cases in which the propagermanium was administered, at the time of starting the administration of the propagermanium and on 35th, 85th, and 357th days after starting the administration. A reduction was observed from the 35th day after starting the administration, and it was confirmed that scar was formed, on 357th day.

These results indicate that medicine of the present invention is useful for treatment of a patient with advanced or recurrent gastric cancer which is non-responsive or non-tolerant to the standard chemotherapy and incurable and unresectable, particularly is useful for metastatic foci.

### <Experimental Example 5> Calculation of Circulating Tumor Cell Index Value in Blood of Oral Cavity Cancer Patient, Gastric cancer Patient, and Healthy Person Before and After Administration of Propagermanium

30 mg of propagermanium [trade name: SEROCION (registered trademark), produced by SANWA KAGAKU KENKYUSHO CO., LTD.] was administered orally in three divided doses per day after meals to 4 patient cases (PG-1 to PG-4) who were diagnosed as advanced or recurrent oral cavity cancer which is non-responsive or non-tolerant to the standard chemotherapy and incurable and unresectable, a 2 patient cases (PG-5 and PG-6) who were diagnosed as advanced or recurrent gastric cancer which is non-responsive or non-tolerant to the standard chemotherapy [gastric cancer treatment guideline fourth edition (edited by Japan Gastric cancer Association, revised in May 2014)] and incurable and unresectable, and 5 healthy person cases (PG-7 to PG-11). 7.5 mL of peripheral blood of the cancer patients (PG-1 and PG-3: only before administration, PG-5 and PG-6: before administration and only 28th day after a start of administration) and healthy persons (all: only before the administration), were collected by a BD vacutainer blood collection tube at the time before the administration of the propagermanium and on 28th and 56th days after starting the administration. Thereafter, and the peripheral blood was centrifuged (1500 G for 15 minutes) to obtain the peripheral blood mononuclear cells (PBMC). The obtained peripheral blood mononuclear cells were fixed and stained using a T cell surface marker and a mouse IgG anti-human antibody against intracellular keratin. The stained cells were analyzed by flow cytometry using a Spectral Cell Analyzer (SP-6800, manufactured by SONY).

Regarding the T cells and the cytotoxic T cells, among living cells, CD45 positive and CD3 positive cells are separated to obtain T cells, and among the CD45 positive and CD3 positive cells, CD8 positive and CD4 negative cells are separated to obtain the cytotoxic T cells. The proportion of the cytotoxic T cells in the T cells was calculated.

In the circulating tumor cells in blood, in order to label the cytokeratin present inside the cells, after using Perm Buffer reagent (#421403, produced by BioLegend), staining was performed using a pankeratin antibody (#4523, produced by Cell Signaling). Since the circulating tumor cells in blood are larger than lymphocytes, when flow cytometry is performed, cells were selected in a wider range than in the selection of normal lymphocytes. After selecting cells negative in staining using Ghost Dye Violet 450 as living cells, pankeratin positive cells were regarded as the circulating tumor cells in blood, and the number of the pankeratin positive cells in 10000 peripheral blood mononuclear cells was calculated.

The calculated number of the pankeratin positive cells and the proportion of cytotoxic T cells in the T cells is substituted into Expression (1) below.

Number of pankeratin positive cells (pieces)/Proportion of cytotoxic T cells in T cells (%)/10 (I)

Then, the tumor immunity risk index value was calculated.

The calculated tumor immunity risk index values are shown in Table 3. The overall survival (OS) is also shown in Table 3 in 4 patient cases with oral cavity cancer (PG-1 to PG-4).

TABLE 3

| | Before | 28th day after | 56th day after | |
|---|---|---|---|---|
| | starting | starting | starting | OS |
| Patient | administration | administration | administration | (day) |
| PG-1 (Oral | 10.53 | — | — | 39 |
| cavity | | | | |
| cancer) | | | | |
| PG-2 (Oral | 0.27 | 1.70 | 4.93 | 105 |
| cavity | | | | |
| cancer) | | | | |
| PG-3 (Oral | 3.50 | — | — | 36 |
| cavity | | | | |
| cancer) | | | | |
| PG-4 (Oral | 2.07 | 0.49 | 1.77 | 85 |
| cavity | | | | |
| cancer) | | | | |
| PG-5 (gastric | 5.04 | 9.55 | — | — |
| cancer) | | | | |
| PG-6 (gastric | 41.12 | 0.31 | — | — |
| cancer) | | | | |
| PG-7 (healthy | 0.60 | — | — | — |
| person) | | | | |
| PG-8 (healthy | 0.60 | — | — | — |
| person) | | | | |
| PG-9 (healthy | 0.60 | — | — | — |
| person) | | | | |
| PG-10 (healthy | 0.43 | — | — | — |
| person) | | | | |
| PG-11 (healthy | 0.21 | — | — | — |
| person) | | | | |

In the oral cavity cancer patient of PG-2, the tumor immunity risk index value before starting the administration of the propagermanium was 0.27. The tumor immunity risk index value on 28th day after starting the administration of the propagermanium was 1.70 and disease was controlled. The administration of the propagermanium was effective.

In the oral cavity cancer patient of PG-4, the tumor immunity risk index value before starting the administration of the propagermanium was 2.07. The tumor immunity risk index values on 28th and 56th days after starting the administration of the propagermanium were 0.49 and 1.77, respectively and the disease was controlled at any time point. The administration of the propagermanium was effective.

In the oral cavity cancer patients of PG-1 and PG-3, from the start of the administration of the propagermanium, the disease got worse and the administration was discontinued midway. Each OS was 39 days and 36 days, and the administration of the propagermanium was ineffective.

On the other hand, in Hyogo College of Medicine Hospital from 2007 to 2016, in 31 cases which were diagnosed as the oral cavity cancer and for which BSC to reduce pain was provided, the median of OS from the time when a doctor explained BSC to the patient was 46 days.

Each OS of the oral cavity cancer patients of PG-2 and PG-4, which are effective examples, were 105 days and 85 days, and it was confirmed that the survival days were extended by the administration of the propagermanium. In the oral cavity cancer patient of PG-4, since the chemotherapy intervention was performed with the disease worsening, the OS was 85 days, before the chemotherapy intervention.

In the gastric cancer patient of PG-5, the tumor immunity risk index value before starting the administration of the propagermanium was 5.04. The tumor immunity risk index value on 28th day after starting the administration of the propagermanium increased to 9.55, and the disease progressed. The administration of the propagermanium was ineffective.

In the gastric cancer patients of PG-6, the tumor immunity risk index value before starting the administration of the propagermanium was 41.12. The tumor immunity risk index value on 28th day after starting the administration of the propagermanium was 0.31, and the disease was controlled. The administration of the propagermanium was effective.

In the gastric cancer patients of PG-S and PG-6, the administration of the propagermanium was discontinued midway at a request of the patient in question.

In addition, in the healthy persons, a median of the tumor immunity risk index value before starting the administration of the propagermanium was 0.60.

From the above, it was confirmed that, when in an evaluation of the therapeutic effect by the medicine including the propagermanium in the cancer patient, it is useful to use the tumor immunity risk index value as an index and the tumor immunity risk index value after completion of experimental administration of propagermanium is 3.5 or less (preferably 3.0 or less) or shows a numerical value less than the tumor immunity risk index value before the administration of the propagermanium, it was evaluated that the therapeutic effect of the medicine including the propagermanium can be expected.

The term “disease is controlled” in the present experimental example indicates that a condition and/or a morbid state of the patient is not deteriorated before and after the administration of the propagermanium. The disease of each patient was determined by an attending physician based on results of endoscopy, computed tomography (CT) examination, positron emission tomography (PET)-CT examination, magnetic resonance imaging (MRI) examination, X-ray (X-P) examination, and blood biochemistry examination including the major markers.

### <Experimental Example 6> Evaluation of Apoptosis-Inducing Activity by Peripheral Blood Mononuclear Cells (PBMC) of Oral Cavity Cancer Patient after Administration of Propagermanium

MKN45 KO cells obtained by fluorescently labeling human gastric cancer cell line MKN45 with Kurabira Orange (1×10⁴ cells) were cultured using an RPMI 1640 medium containing 10% FBS in 35 mm dishes (Day 0). On next day (Day 1), the medium was changed to an F12-K medium (2 mL) containing 10% FBS, and in Experimental Example 5, a suspension of peripheral blood mononuclear cells (1×10⁶ cells) separated from peripheral blood of the oral cavity cancer patient of PG-4 before the administration of the propagermanium and on 28th and 56th days after the administration in F12-K medium (1 mL) is added to the culture dish. Furthermore, 3 μL of IncuCyte (registered trademark) Caspase-3/7 Green Apoptpsis Reagent (Cat No. 4440, produced by Essen BioScience) was added and cultured. Time-lapse photography was performed every 10 minutes for 48 hours, by using BioStation (produced by Nikon Corporation) (Days 1 to 3). An animation was created from result of photography for 48 hours, and the numbers of viable gastric cancer cells (with orange fluorescence) at and apoptosis-induced gastric cancer cells (with green fluorescence) at the time points of 0, 8, 16, 24, 32, 40, and 48 hours were calculated respectively. When calculating the number of cells, 100 to 150 cells were targeted at time point of 48 hours, and cells of 4 to 5 fields of view were summed at 200 times observation by a microscope at each time point. In addition, at each time point, only cells that adhered to the culture dish and could be continuously observed were targeted for counting, and cells that were slightly out of an imaging field of stratified cells were excluded from counting.

The apoptosis-inducing activity (%) at each time point was calculated by using Expression (II) below.

{Number of apoptosis-induced gastric cancer cells/(Number of viable gastric cancer cells+Number of apoptosis-induced gastric cancer cells)}×100 (II)

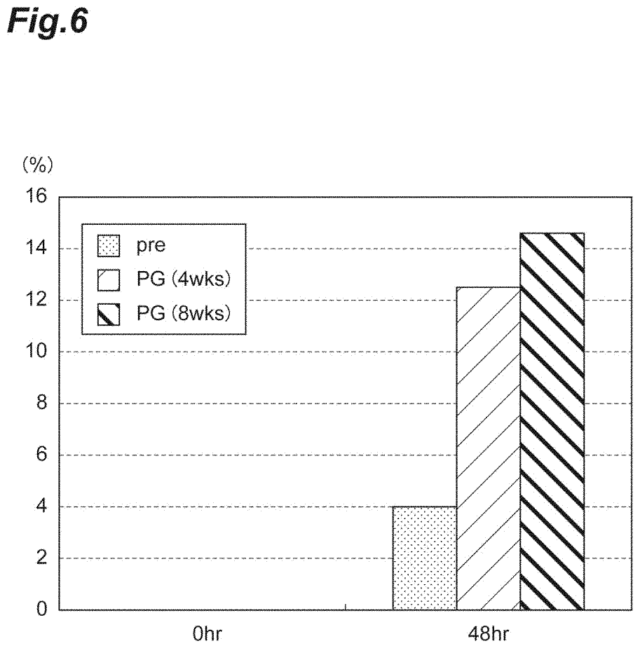

The calculated apoptosis-inducing activity is shown in Table 4 and FIG. 6.

TABLE 4

| | Apoptosis-inducing activity (%) | |
|---|---|---|
| | Culture for 0 hours | Culture for 48 hours |
| Before starting | 0.00 | 4.00 |
| administration | | |
| 28th day after starting | 0.00 | 12.50 |
| administration | | |
| 56th day after starting | 0.00 | 14.60 |
| administration | | |

At the time point of 48 hours, the apoptosis-inducing activity by the peripheral blood mononuclear cells of the oral cavity cancer patient who received the propagermanium by administration increased approximately 3.1 times at 28th day after starting the administration and approximately 3.7 times at 56th day after starting the administration, compared to activity before starting the administration. That is, it was confirmed that the apoptosis-inducing activity of the peripheral blood mononuclear cells to cancer cells increased by the administration of the propagermanium. It was confirmed that the apoptosis-inducing activity was not specific to a specific cancer cell, and showed the apoptosis-inducing activity to different kinds of cancer cells.

1.-12. (canceled)

13. A method for treating a patient with advanced or recurrent cancer which is non-responsive or non-tolerant to standard chemotherapy and incurable and unresectable, the method comprising: administering propagermanium to the patient in need thereof.

14. The method according to claim 13,
wherein the cancer patient is an adenocarcinoma patient.

15. The method according to claim 14,
wherein the adenocarcinoma patient is a patient with one or more kinds of cancer selected from the group consisting of gastric cancer, biliary tract cancer, liver cancer, pancreatic cancer, prostate cancer, breast cancer, ovarian cancer, colon cancer, and rectal cancer.

16. The method according to claim 13,
wherein the adenocarcinoma patient is a gastric cancer patient.

17. The method according to claim 16,
wherein the standard chemotherapy is standard chemotherapy using one or more chemotherapeutic agents selected from the group consisting of S-1, capecitabine, 5-fluorouracil, cisplatin, oxaliplatin, docetaxel, paclitaxel, irinotecan, trastuzumab, and ramucirumab.

18. The method according to claim 13,
wherein the cancer patient is a squamous cell carcinoma patient.

19. The method according to claim 18,
wherein the squamous cell carcinoma patient is a patient with one or more kinds of cancer selected from the group consisting of oral cavity cancer, pharyngeal cancer, laryngeal cancer, thyroid cancer, salivary gland cancer, esophageal cancer, lung cancer, skin cancer, and uterine cancer.

20. The method according to claim 18,
wherein the squamous cell carcinoma patient is an oral cavity cancer patient.

21. The method according to claim 20,
wherein the standard chemotherapy is standard chemotherapy using one or more chemotherapeutic agents selected from the group consisting of S-1, 5-fluorouracil, cisplatin, carboplatin, nedaplatin, docetaxel, paclitaxel, and cetuximab.

22. The method according to claim 13,
wherein the cancer patient is a patient whose ECOG performance status is 0 or 1.

23. The method according to claim 13,
wherein the cancer patient is a patient without peritoneal metastasis.

24. The method according to claim 13,
wherein the cancer patient is a patient whose a tumor immunity risk index value after completion of experimental administration of the propagermanium is 3.5 or less or shows a numerical value less than the tumor immunity risk index value before the experimental administration of the propagermanium.
